(19) United States
(12) Patent Application Publication
(10) Pub. No.: US 2023/0112422 A1
(43) Pub. Date: Apr. 13, 2023

(54) IMPROVEMENTS RELATING TO PROVISION OF GAS-FLOW

(71) Applicant: Fisher & Paykel Healthcare Limited, Auckland (NZ)

(72) Inventor: Matthew Jon Payton, Auckland (NZ)

(21) Appl. No.: 17/904,898

(22) PCT Filed: Feb. 26, 2021
(86) PCT No.: PCT/IB2021/051587

§ 371 Date: Aug. 24, 2022

Related U.S. Application Data
(60) Provisional application No. 62/982,298, filed on Feb. 27, 2020.

(51) Int. Cl. A61M 16/16

(52) U.S. Cl. CPC A61M 16/16; A61M 2016/0027

(57) ABSTRACT

The disclosure relates to a method and respiratory system, comprising: a flow generator to provide a gas-flow to a patient, the gas flow comprising an oxygen fraction, and a controller configured to: receive input relating to oxygen fraction at a patient's nose and/or mouth, adjust the gas-flow flow rate based on the oxygen fraction at the patient's nose and/or mouth.

### FIELD OF THE INVENTION

The present invention relates to a method and apparatus for determining whether a respiratory system is providing a sufficient flow of gas to meet a patient's inspiratory demand and/or providing a gas flow to meet or at least get closer to a patient's inspiratory demand.

### BACKGROUND TO INVENTION

High flow respiratory systems are used to provide a flow of gas with a high flow rate to a patient and with an oxygen concentration for respiratory support. For example, such a flow of gas might be provided to assist with patient breathing and/or oxygenate a patient. Oxygenation of the patient may be desirable in various circumstances, such as where the lung function of a patient is diminished and needs assistance, for example when the patient is suffering from a respiratory disease or disorder, in intensive care, or when the patient's oxygen reservoir needs to be increased prior to an anaesthetic procedure (called “pre-oxygenation”). The term “anaesthetic procedure” can be used to cover general anaesthesia, procedural sedation and regional/local anaesthesia. There could be other uses of high flow respiratory systems too, and the embodiments herein could be used with any of the other applications of a high flow respiratory system.

To provide effective respiratory support, the high flow respiratory system should provide a sufficiently high flow rate such that the inspiratory demand, and preferably patient peak inspiratory demand, is met. However, it should be noted that there is considerable inter-patient and intra-patient variability in peak inspiratory demand caused by a variety of factors including anatomy, physiology, anxiety, level of consciousness, and respiratory disease state. Such variabilities makes it more difficult to determine whether the high flow respiratory system is providing a gas flow at a suitable flow rate for meeting the patient's inspiratory demand as providing a “high” flow rate may not be sufficiently high enough to meet the inspiratory demand of the patient. Further, it is important to minimise or avoid patient discomfort associated with excessively high flow rates.

It is therefore desirable to provide a method and apparatus for determining whether a respiratory system is providing flow of gas at a flow rate that meets an inspiratory demand of a patient, such as a peak inspiratory demand.

### SUMMARY OF INVENTION

It is an object of the present invention to provide gas-flow to a patient on the basis of inspiratory demand, and/or to obtain an indication of inspiratory demand. For example, embodiments determine whether a respiratory system is providing a flow of gas that meets a patient's inspiratory demand. This could be, for example, instantaneous inspiratory demand or peak inspiratory demand.

In one aspect the present invention may be said to comprise a respiratory system, comprising: a flow generator to provide a gas-flow to a patient, the gas flow comprising an oxygen fraction, and a controller configured to: receive input from a sensor, adjust the gas-flow flow rate based on an oxygen fraction at the patient's nose and/or mouth.

In another aspect the present invention may be said to comprise a respiratory system, comprising: a flow generator to provide a gas-flow to a patient, the gas flow comprising an oxygen fraction, and a controller configured to: receive input relating to oxygen fraction at an patient's nose and/or mouth, adjust the gas-flow flow rate based on the oxygen fraction at the patient's nose and/or mouth.

Optionally the flow generator provides a high flow gas-flow.

Optionally the controller is further configured to obtain an indication of the oxygen fraction at the patient's nose or mouth.

Optionally the gas-flow rate is adjusted based on a relationship between the oxygen fraction the patient's nose and/or mouth and the gas-flow oxygen fraction.

Optionally the sensor is a O2 fraction sensor coupled to the controller.

Optionally the system further comprises one or more of:
a humidifier for humidifying the gas-flow,
inspiratory tube,
conduit (e.g. dry line or heated breathing tube),
patient interface,
pressure relief valve
filter.

Optionally the controller is further configured to: determine if the gas-flow meets inspiratory demand or does not meet inspiratory demand based on the oxygen fraction at the patient's mouth and/or nose.

Optionally the controller is further configured to: determine if the gas-flow meets inspiratory demand or does not meet inspiratory demand based on a relationship between oxygen fraction at the patient's mouth and/or nose and the gas-flow oxygen fraction.

Optionally the system further comprises a user interface and the controller configured to convey to a user whether a patient is meeting or not meeting inspiratory demand.

Optionally the oxygen fraction is: at least more than about 21%, and optionally: 100%, or between about 30% and about 50%.

Optionally the flow rate is about 20 litres per minute or more, optionally between about 20 litres per minute and 90 litres per minute, and optionally between about 40 litres per minute and 70 litres per minute.

Optionally the system comprising or for use with a non-sealing patient interface, preferably a non-sealing nasal cannula.

Optionally whether the gas-flow meets the patient's inspiratory demand comprises comparing the oxygen fraction at the mouth and/or nose against the gas-flow oxygen fraction.

Optionally if the determined oxygen fraction at the patient's nose and/or mouth is less than the gas-flow oxygen fraction, then it is determined that the gas-flow does not meet the patient's inspiratory demand.

Optionally if oxygen fraction at the patient's nose and/or mouth is equal to or about (“matches”) the gas-flow oxygen fraction, then it is determined that the gas-flow meets or is close to the patient's inspiratory demand.

Optionally if the inspiratory demand is not being met, the gas-flow flow rate is increased by the controller.

Optionally the gas-flow flow rate is increased to a flow rate of about 20 litres per minute or more, optionally between about 20 litres per minute and about 90 litres per minute, or between about 40 litres per minute and about 70 litres per minute.

Optionally if the inspiratory demand is being exceeded, the gas-flow flow rate is maintained or decreased by the controller.

Optionally the gas-flow flow rate is maintained until it is determined that the patient inspiratory demand is not being met.

Optionally determination that patient inspiratory demand is not being met comprises the controller: monitoring a set number of previous patient breath cycles, determining a number of previous patient breath cycles that have entrainment of ambient air, and comparing the number of previous patient breath cycles having entrainment of ambient air against a set threshold.

Optionally patient inspiratory demand is not met if the number of previous patient breath cycles having entrainment of ambient air exceeds a set threshold.

Optionally the system is operated to provide the gas-flow to a patient prior to an anaesthetic procedure, and the oxygen fraction of the gas-flow is 100%.

Optionally the system is operated to provide the gas-flow to a patient during a sedation procedure, and the oxygen fraction of the gas-flow is about 21% or more.

Optionally during the sedation procedure, the oxygen fraction of the gas-flow is between about 21% and about 100%.

In another aspect the present invention may be said to comprise a method of providing a flow of gas from a respiratory system to a patient, comprising the steps of: providing a gas-flow to a patient comprising an oxygen fraction, adjusting the gas-flow flow rate based on an oxygen fraction at the patient's nose and/or mouth.

In another aspect the present invention may be said to comprise a method of providing a flow of gas from a respiratory system to a patient, comprising the steps of: providing a gas-flow to a patient comprising an oxygen fraction, and determining from an oxygen fraction at the patient's mouth and/or nose whether the gas-flow meets the patient's inspiratory demand.

Optionally the gas-flow is high flow.

Optionally the method further comprises obtaining an indication of the oxygen fraction at the patient's nose or mouth.

Optionally the gas-flow rate is adjusted based on a relationship between the oxygen fraction the patient's nose and/or mouth and the gas-flow oxygen fraction.

Optionally determining whether the gas-flow meets the patient's inspiratory demand is based on a relationship between the oxygen fraction at the patient's mouth and/or nose against the gas-flow oxygen fraction.

Optionally if the oxygen fraction at the patient's nose and/or mouth is less than the gas-flow oxygen fraction, then it is determined that the gas-flow does not meet the patient's inspiratory demand.

Optionally if the oxygen fraction at the patient's nose and/or mouth is equal to or about (“matches”) the gas-flow oxygen fraction, then it is determined that the gas-flow meets the patient's inspiratory demand.

Optionally if the inspiratory demand is not being met, the gas-flow flow rate is increased.

Optionally the gas-flow flow rate is increased to a flow rate of about 20 litres per minute or more, optionally between about 20 litres per minute and about 90 litres per minute, or between about 40 litres per minute and about 70 litres per minute.

Optionally if the inspiratory demand is being exceeded, the gas-flow flow rate is maintained or decreased.

Optionally the gas-flow flow rate is maintained until it is determined that the patient inspiratory demand is not being met.

Optionally determination that patient inspiratory demand is not being met comprises the steps of: monitoring a set number of previous patient breath cycles, determining a number of previous patient breath cycles that have entrainment of ambient air, and comparing the number of previous patient breath cycles having entrainment of ambient air against a set threshold.

Optionally patient inspiratory demand is not met if the number of previous patient breath cycles having entrainment of ambient air exceeds a set threshold.

Optionally gas-flow is provided to the patient prior to an anaesthetic procedure, and the oxygen fraction of the gas-flow is 100%.

Optionally gas-flow is provided to the patient during a sedation procedure, and the oxygen fraction of the gas-flow is about 21% or more.

Optionally during the sedation procedure, the oxygen fraction of the gas-flow is between about 21% and about 100%.

Optionally a controller and flow generator to adjust the gas-flow flow rate based on an oxygen fraction at the patient's nose and/or mouth and oxygen fraction of the gas-flow to meet or get close to inspiratory demand.

In another aspect the present invention may be said to comprise a method of adjusting a flow rate of gas-flow provided to a patient receiving high gas flows, the method comprising: a) providing a gas-flow at high flow rates to a patient, the gases flow comprising a target gas; b) obtaining a target gas measurement from the patient's nose and/or mouth; c) adjusting the flow rate of the gases flow based on the target gas measurement.

In another aspect the present invention may be said to comprise a system for adjusting a flow rate of a gas flow provided to a patient receiving high gas flows, the system comprising: flow source configured to provide a gas flow at high flow rates to a patient, the gas flow comprising a target gas; a controller configured to receive an input comprising a target gas measurement from the patient's nose and/or mouth; wherein the controller is configured to adjust the flow rate of the gas flow based on the target gas measurement.

The method and/or system might have the following features.

Optionally the method further comprises reiterating steps, or the controller is configured to reiterate the steps, a) to c) until a desired target gas measurement is obtained.

Optionally if the target gas measurement is less than the desired target gas measurement, the flow rate of gas flow is increased until the desired target gas measurement is obtained

Optionally if the target gas measurement is more than the desired target gas measurement, the flow rate of gas flow is decreased until the desired target gas measurement is obtained.

Optionally target gas comprises O2.

Optionally a) comprises providing 100% of O2/an O2 fraction of 1 to the patient at high flow rates.

Optionally the target gas measurement comprises a measured O2 fraction.

Optionally the method further comprises the step of optimising or the controller is configured to optimise the flow rate of the gas-flow to meet the patient's peak inspiratory demand.

Optionally the desired target gas measurement comprises a measured O2 fraction of about or close to 100%/O2 fraction of about or close to 1.

Optionally if the measured O2 fraction is substantially less than 100%/a fraction of 1, the flow rate of gas-flow is increased until a measured O2 fraction of about or close to 100%/a measured O2 fraction of about or close to 1 is obtained.

Optionally if the measured O2 fraction is 100%/a fraction of 1, the flow rate of gas-flow is decreased until a measured O2 fraction of about or close to 100%/a measured O2 fraction of about or close to 1 is obtained Optionally, the flow rates are about 20 L per minute or more.

Optionally the flow rate are about 20 L per minute to about 90 L per minute.

Optionally the gas flow is humidified.

Optionally the patient is spontaneously breathing.

Optionally the patient is undergoing preoxygenation prior to an anaesthetic procedure.

In another aspect the present invention may be said to comprise a method of determining whether peak inspiratory demand of a patient is met, the method comprising: a) providing a gas-flow at high flow rates to a patient, the gas-flow comprising a target gas at a gas fraction (such as 100%/1); b) measuring a target gas fraction at or close to the patient's nose and/or mouth; wherein if the measured target gas fraction is substantially less than 100%/a fraction of 1, the patient's peak inspiratory demand is not met, and wherein if the measured target gas fraction is about or close to 100%/about or close to a fraction of 1 (that is, almost or up to but nor more than), the patient's peak inspiratory demand is substantially met wherein if the measured target gas fraction is 100%/a fraction of 1, the patient's peak inspiratory demand is met or exceeded.

In another aspect the present invention may be said to comprise a system for determining whether peak inspiratory demand and a patient is met, the system comprising: a flow source configured to provide a gases flow at high flow rates to a patient, the gases flow comprising a target gas at 100%/a target gas fraction of 1; a controller configured to receive an input comprising a target gas fraction measurement at or close to the patient's nose and/or mouth; the controller configured to provide an output relating to the peak inspiratory demand of a patient, wherein if the measured target gas fraction is substantially less than 100%/a fraction of 1, the patient's peak inspiratory demand is not met, and
wherein if the measured target gas fraction is about or close to 100%/about or close to (that is, almost or up to but nor more than) a fraction of 1, the patient's peak inspiratory demand is substantially met
wherein if the measured target gas fraction is 100%/a fraction of 1, the patient's peak inspiratory demand is met or exceeded.

The method and/or system might have the following features.

Optionally the method comprises steps of providing, or the controller is configured to provide, an output on whether patient's respiratory demand is met.

Optionally the output is provided on display.

Optionally the method comprises the steps of adjusting, or the controller is configured to adjust, the flow rate of the gas-flow to substantially meet the patient's peak inspiratory demand.

Optionally, the flow rates are about 20 L per minute or more.

Optionally the flow rates are about 20 L per minute to about 90 L per minute.

Optionally the gas flow is humidified.

Optionally the patient is spontaneously breathing.

Optionally the patient is undergoing preoxygenation prior to an anaesthetic procedure.

In this specification, “high flow” means, without limitation, any gas flow with a flow rate that is higher than usual/normal, such as higher than the normal inspiration flow rate of a healthy patient. Alternatively or additionally, it can be higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult, child) and the like. Those skilled in the art know from context what constitutes “high flow”. It is a magnitude of flow rate that is over and above a flow rate that might otherwise be provided.

But, without limitation, some indicative values of high flow can be as follows.
In some configurations, delivery of gases to a patient at a flow rate of greater than or equal to about 5 or 10 litres per minute (5 or 10 LPM or L/min).
In some configurations the flow rate is about 20 litres per minute or more, optionally between about 20 litres per minute and 90 litres per minute, and optionally between about 40 litres per minute and 70 litres per minute.
In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, bout 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

High flow has been found effective in meeting or exceeding the patient's normal real inspiratory flow, to increase oxygenation of the patient and/or reduce the work of breathing. Additionally, high flow therapy may generate a flushing effect in the nasopharynx such that the anatomical dead space of the upper airways is flushed by the high incoming gas flows. This creates a reservoir of fresh gas available for each and every breath, while minimising re-breathing of carbon dioxide, nitrogen, etc.

By example, a high flow respiratory system 10 is described with reference to FIG. 3. High flow may be used as a means to promote gas exchange and/or respiratory support through the delivery of oxygen and/or other gases, and through the removal of CO2 from the patient's airways. High flow may be particularly useful prior to, during or after a medical and/or anaesthetic procedure.

When used prior to a medical procedure, high gas flow can pre-load the patient with oxygen so that their blood oxygen saturation level and volume of oxygen in the lungs is higher to provide an oxygen buffer while the patient is in an apnoeic phase during the medical and/or anaesthetic procedure.

A continuous supply of oxygen is essential to sustain healthy respiratory function during medical procedures (such as during an anaesthetic procedure) where respiratory function might be compromised (e.g. diminishes or stops). When this supply is compromised, hypoxia and/or hypercapnia can occur. During anaesthetic procedures such as general anaesthesia where the patient is unconscious, the patient is monitored to detect when this happens. If oxygen supply and/or CO2 removal is compromised, the clinician stops the medical procedure and facilitates oxygen supply and/or CO2 removal. This can be achieved for example by manually ventilating the patient through an anaesthetic bag and mask, or by providing a high flow of gases to the patient's airway using a high flow respiratory system.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO2.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.”

In this specification, “oxygen concentration” may be referred to in terms of an “oxygen fraction”. For example, the oxygen concentration of ambient air is an oxygen fraction of 21% (this can be expressed as 0.21). In another example, the oxygen concentration of pure air is an oxygen fraction of 100% (this can be expressed as 1). The terms “oxygen fraction” and “oxygen concentration” can be used interchangeably.

In this specification, “inspiratory demand” refers to the flow rate of gas the patient inspires.

In this specification, “(patient) peak inspiratory demand” refers to the peak flow rate of gas inspired by the patient. Peak inspiratory demand is met when a flow rate of gas is provided to a patient at a flow rate substantially equal to or more than the peak inspiratory demand.

In this specification, “(patient) instantaneous inspiratory demand” refers to the flow rate of gas inspired by a patient at an instant point in time. Instantaneous inspiratory demand is met when a flow rate of gas is provided to a patient at a flow rate substantially equal to or more than the instantaneous inspiratory demand.

In this specification, the term “meeting” in the context of “meeting inspiratory demand” or “meeting a flow rate” or similar means equal to, or close to or otherwise within some suitable tolerance. The tolerance will be defined by that which still achieves the advantages of the embodiments described, and could be for example (but by way of example only and without limitation) +/−10%, +/−9%, +/−8%, +/−7%, +/−6%, +/−5%, +/−4%, +/−3%+/−2%, +/−1%, 0% or some fraction in between those numbers. “Meeting” does not mean that it has to be an exact match. Furthermore, “meeting” in this context could also mean “at least meeting”, which could mean that the inspiratory demand (that is, demand flow rate) is actually exceeded. As will be seen, many cases it is not known whether the inspiratory demand (that is, demand flow rate) is met or exceeded, but that does not detract from the advantages—the factor of knowing that it is one of meeting or exceeding is sufficient.

In this specification, the term “equal” or similar terms in the context of “a measured concentration of oxygen at the patient being equal to the concentration of oxygen being delivered” or similar means at, or close to or otherwise within some suitable tolerance. The tolerance will be defined by that which still achieves the advantages of the embodiments described and could be for example (but by way of example only and without limitation) +/−10%, +/−9%, +/−8%, +/−7%, +/−6%, +/−5%, +/−4%, +/−3%+/−2%, +/−1%, 0% or some fraction in between those numbers. “Equal” does not mean that it has to be an exact match.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

In this specification where reference has been made to patent specifications, other external documents, or other sources of information, this is generally for the purpose of providing a context for discussing the features of the disclosure. Unless specifically stated otherwise, reference to such external documents is not to be construed as an admission that such documents, or such sources of information, in any jurisdiction, are prior art, or form part of the common general knowledge in the art.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth. The invention consists in the foregoing and also envisages constructions of which the following gives examples only.

### BRIEF DESCRIPTION OF DRAWINGS

Embodiments will be described with reference to the following Figures, of which:

FIG. 1 shows a representation of a patient breath.

FIG. 2A shows a flow of gas to a patient and entrainment air, where the flow of gas does not meet peak inspiratory demand.

FIG. 2B shows a flow of gas to a patient without entrainment of air, when the flow of gas meets peak inspiratory demand.

FIG. 3 shows a respiratory system for determining peak inspiratory demand.

FIG. 4 shows an operation of the respiratory system for determining peak inspiratory demand.

FIG. 5 shows a flow that may meet inspiratory demand over a portion of a patient's breath but may not meet the patient's peak inspiratory demand and a flow that meets the patient's inspiratory demand and peak inspiratory demand.

FIG. 6 shows a composition of flow inspired by the patient.

FIG. 7 shows fraction of oxygen measured.

### DETAILED DESCRIPTION

Overview

There are a number of clinical situations where delivering a known fraction of inspired oxygen (that is “oxygenation”) is important (reference herein to “fraction” can be used interchangeably with the terms “concentration” and “proportion”). For example, during an oxygenation phase (commonly referred to as pre-oxygenation) before general anaesthesia, it is desirable to administer a fraction of inspired oxygen to ensure that the lungs contain as much oxygen as possible. A further example is that it is desirable to administer a known fraction of inspired oxygen to patients being treated for respiratory distress. In another more general example, respiratory support might be being provided to a patient—e.g. respiratory support provided to a patient using a gas flow with a high flow rate (“high flow”). In the clinical situation, the patient might be spontaneously breathing, or might not be spontaneously breathing—depending on the situation.

Where it is desirable to deliver a flow of gas to oxygenate (that is to deliver oxygen to meet the oxygen needs of) a patient, the flow of gas can be provided with desired oxygen concentration (that is usually above the oxygen concentration of ambient air). For example, this might be in situations where ambient air cannot meet the oxygenation needs of the patient, as ambient air may not have a high enough oxygen concentration to effectively oxygenate the patient. This can be achieved by operating a respiratory system to deliver a flow of gas with an oxygen concentration that is higher than the oxygen concentration found in ambient air such that the flow of gas meets the oxygenation needs of the patient.

For the flow of gas to effectively oxygenate the patient, the flow of gas should have an oxygen concentration required by the patient and a flow rate that meets the inspiratory demand of the patient. In the case of peak inspiratory demand the flow rate required to meet demand is the peak flow rate of gas that the patient inspires during a breath cycle. In the case of instantaneous demand, the flow rate required at a particular time to meet demand is the flow rate that the patient inspires at that particular time.

If the flow rate of gas being delivered to a patient does not meet inspiratory demand, then entrainment of ambient air can occur. Entrainment of ambient air may occur via the patient's nose and/or mouth. Entrainment of ambient air could occur via the patient's mouth if the patient's mouth is open. If this occurs, the concentration of the constituent gas will be altered (usually diluted) due to the different concentration of that constituent gas in ambient air. This means the intended concentration of the constituent gas is not actually provided.

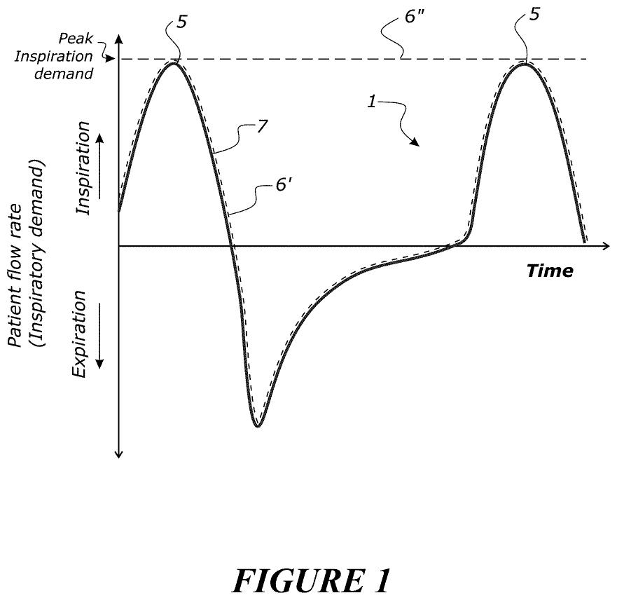

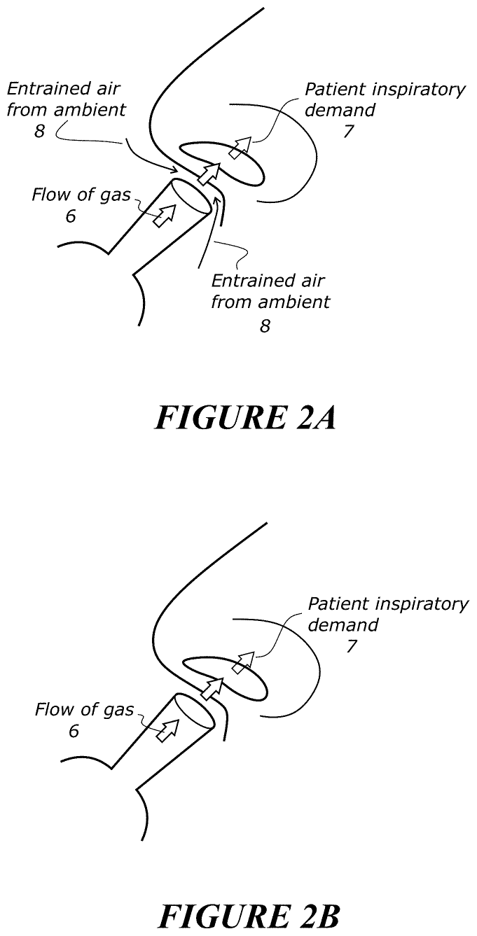

This will be explained further with reference to FIG. 1 that shows a line graph showing the breath flow 1 (and therefore the inspiratory demand 7) of a patient and how it changes over the course of a patient's breath cycle, by way of example. The inspiratory demand varies with respect to time (instantaneous inspiratory demand 7), and reaches a peak inspiratory demand 5. When delivering a desired oxygen concentration to a patient, it is desirable that the flow rate of the flow of gas 6 from the respiratory system is the same (be it peak 6″ or instantaneous 6′) as the inspiratory demand 7 flow rate, otherwise the patient entrains ambient air 8 during inspiration, as shown in FIG. 2A (Note, while only the nose is shown in FIG. 2A for simplicity, it be noted that entrainment can happen through the nose and/or mouth).

As the concentration of oxygen in ambient air is less than in the flow of gas provided from the respiratory system, entrainment of ambient air is undesirable because it dilutes and lowers the oxygen concentration (and therefore level of oxygenation) received by the patient. Conversely, if the flow rate of the gas provided from the respiratory system meets inspiratory demand flow rate, then the patient only inspires the flow of gases provided by the respiratory system (that is, does not entrain ambient air), and therefore receives the desired oxygen concentration, as provided by the flow of gas. This happens when the respiratory system delivers a flow of gas at a flow rate that is greater than or equal to the inspiratory demand of the patient.

To avoid or reduce entrainment, the flow rate from the nasal high flow therefore desirably meets or exceeds the patient's inspiratory demand (that is, the flow rate that is being inspired). If the patient's inspiratory demand is met by the flow rate of the gases flow being provided, the patient may not entrain ambient air.

This could be achieved by meeting peak inspiratory demand—by meeting peak inspiratory demand, the gases flow 6 may be provided to the patient at a constant high flow flow rate (see horizontal dotted line 6″ in FIG. 1) at or greater than the peak inspiratory demand 5, which means it will at least meet, and will usually exceed, instantaneous inspiratory demand at any particular part of the breath cycle. That is, if gas-flow flow rate is always meeting peak inspiratory demand, then it will be meeting the demand at any point in the cycle (instantaneous inspiratory demand—assuming gas flow 6 is constant) because the instantaneous inspiratory demand will never be more than the peak inspiratory demand.

Alternatively, this could be achieved by meeting or exceeding instantaneous inspiratory demand 7 at any point in time—see curved dotted line 6′. Reference herein to “inspiratory demand” can be used to encompass either peak inspiratory demand or instantaneous inspiratory demand. Reference numeral 6 can be used to generally refer to flow of gas provided. Reference number 6″ refers to a gas flow that meets peak inspiratory demand. Reference number 6′ can refers to a changing gas flow that meets instantaneous inspiratory demand (of which gas flow 6″ that meets peak inspiratory demand is a special case).

There is considerable inter-patient and intra-patient variability in inspiratory demand caused by a variety of factors including anatomy, physiology, anxiety, level of consciousness, and respiratory disease state. Therefore, determining and providing a gases flow with a flow rate sufficient to meet inspiratory demand can be difficult. One solution would be to provide a significantly high flow rate that would be certain to meet the patient's inspiratory demand (peak or instantaneous). But, having significantly high flow rates of gas flow in excess of the inspiratory demand could cause discomfort to the patient. It is therefore desirable to deliver suitable flow rate or range of flow rates to the patient to meet inspiratory demand (be that peak inspiratory demand or instantaneous inspiratory demand) without providing unnecessarily high flow rates that unduly exceed the inspiratory demand.

In general terms, without limitation, embodiments described here achieve this by placing a suitable sensor, e.g. a gas sampling device, such as a cannula with gas sampler attachment (see for example U.S. patent application 62/408,480 and U.S. patent application 62/492,783 which are incorporated herein by reference in its entirety), at the patient's mouth and/or nose. If the sampler measures less (or different, such as higher, but typically it will be less as delivered gas concentration will typically be higher than ambient) than the gas concentration being delivered, then the inspiratory demand is not being met and the flow rate of the gases provided to the patient needs to be increased. If the patient's inspiratory demand is being met, then the gas sampler will measure a concentration of oxygen equal to (see definition above, which means this also covers being at, close to, or within some tolerance of) the concentration of oxygen being delivered. In this case, the flow rate of the gases provided to the patient can be maintained. So, it can be determined if the gas-flow meets inspiratory demand, or does not meet inspiratory demand, based on a determined oxygen fraction (either by the controller and/or a sensor). Adjustment of the flow rate can then be made depending on whether gas flow meets inspiratory demand (maintain or decrease flow rate) or does not meet inspiratory demand (gas-flow increased). To do this, the controller in the respiratory system receives input relating to the gas concentration (e.g. concentration of oxygen) at the nose and/or mouth of the patient. This could be input received directly from a sensor, or input received from a user who inputs measurements being or relating to gas concentration. Possibly, the user ascertains that measurement from a sensor, although they might ascertain that information in other ways. In some embodiments, the controller might receive information either from a sensor, user or other source about whether the gas concentration at the patient nose and/or mouth meets the delivered gas concentration, and use that information to control the respiratory system flow rate. That is, the controller does not determine whether the gas concentration of the nose and/or mouth meets the delivered gas concentration, it simply receives input regarding that determination. Any reference to sensing gas fraction at a patient mouth and/or nose herein could be read as meaning directly sensed and input from a sensor to the respiratory apparatus/controller, or sensed but then received indirectly in another way, such as input by a user who e.g. has read a sensor output. Receiving input from a sensor can be considered to cover both options—that is, both direct and indirect input from a sensor.

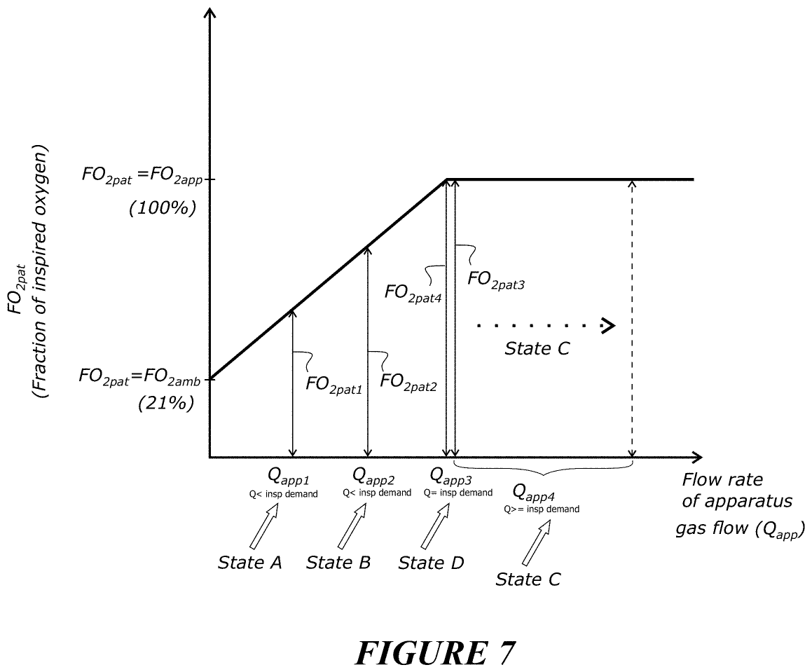

Referring to FIG. 7, when the concentration of oxygen measured is equal to concentration of oxygen being delivered, it could in fact be due to the fact that the flow rate is in excess of the patient's inspiratory demand. In this case, it might be desirable to decrease the flow rate being delivered, so as not to provide unnecessarily excessive therapy which may lead to discomfort or other undesirable outcomes (e.g. O2 wastage). In this case, it is possible to titrate the flow rate provided to the patient in the following way to get closer to the actual inspiratory demand (rather than exceeding it). The flow rate can be decreased, and then another measurement taken. If the measured oxygen concentration is still equal to the delivered concentration, inspiratory demand is still at least being met (or possibly exceeded), so the flow rate is decreased again. This can be continued until the measured oxygen concentration is less than that delivered oxygen concentration, which indicates that patient is entraining air, and so this will be just below the point of meeting inspiratory demand. Optionally, at this point the flow rate can be increased again to finally settle on or close to the inspiratory demand. Therefore, the flow rate is decreased until the oxygen concentration level reaches or is close to the optimal value. The flow rate provided to the patient can therefore be titrated based on whether the patient's inspiratory demand is met, where the patient's inspiratory demand may be determined based on the concentration of oxygen inspired by the patient (also known as fraction of inspired oxygen). More sophisticated control techniques may be applied.

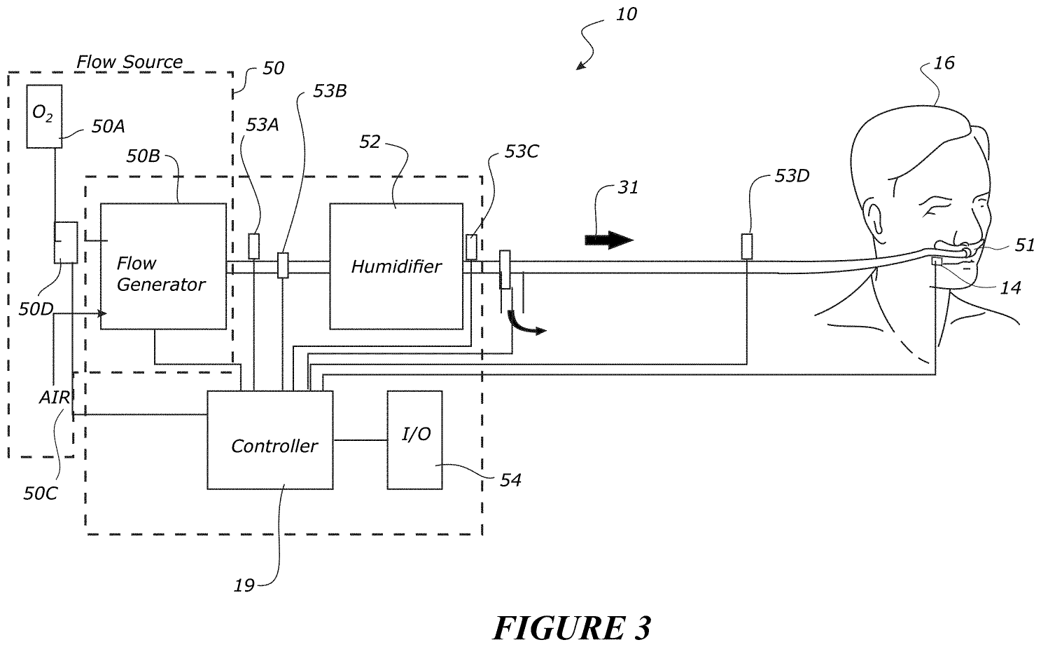

FIG. 3 shows a respiratory system that can provide a flow of gas (with a known oxygen fraction) for providing respiratory support (preferably oxygenation) to a patient, determine whether the flow of gas has a flow rate that meets the patient's inspiratory demand (that is, the flow rate of the gases inspired by the patient), and optionally adjust the flow rate of the flow of gas 6 so that it does meet the inspiratory demand flow rate (“inspiratory demand”), or any other flow rate as required. In one option, the respiratory system determines whether the flow of gases has a flow rate 6 that meets the patient's peak inspiratory demand 5 (that is, the flow rate of the gases inspired by the patient at peak inspiration), and optionally adjust the flow rate of the flow of gas 6″ so that it does meet the peak inspiratory demand flow rate (“peak inspiratory demand”), or any other flow rate as required. This is achieved by measuring the O2 concentration at the nose and/or mouth at the time of peak inspiratory demand, or throughout the entire cycle. Alternatively, the respiratory system could be configured to determine whether the flow rate being 6 provided is meeting instantaneous inspiratory demand 7 (that is, the flow rate of gas inspired by the patient at an instant point in time) at any part of the inspiration cycle. This is achieved by measuring the O2 concentration at the nose and/or mouth continuously or periodically throughout the breath cycle. Optionally it adjusts the flow rate 6′ of the flow of gas so that it does meet the instantaneous inspiratory demand flow rate (“instantaneous inspiratory demand”), or any other flow rate as required. Alternatively or additionally, and more generally, the respiratory system could adjust the gas-flow flow rate 6 based on a determined oxygen fraction or other parameter at the patient's nose and/or mouth. For example, a controller can be configured to receive input relating to an oxygen fraction at the patient's nose and/or mouth, and adjust the gas-flow flow rate based on the oxygen fraction at the patient's mouth and/or nose. In any variation, the measurement of O2 concentration can be provided as input to the respiratory system direct from a sensor, or indirectly such as provided by a user that reads a sensor.

Preferably the option where the respiratory system is configured to determine whether the flow rate being provided is meeting peak inspiratory demand 5 will be used. If peak inspiratory demand is determined and met, then it will follow that any inspiratory demand 7 at any other part of the breathing cycle will be met (assuming the flow rate 6″ is kept constant—see FIG. 1 dotted horizontal line).

Of course, in an alternative, the instantaneous inspiratory demand 7 is determined, periodically or continuously and the apparatus flow rate 6′ changed (titrated) periodically or continuously to meet that instantaneous inspiratory demand 7 (see dotted curved line 6′ in per FIG. 1).

In the embodiments described below, such determination is achieved by comparing the oxygen fraction of the gas inspired by the patient (or some proxy measurement of that concentration or determined in some other way) against the oxygen fraction or concentration of the flow of gas provided by the respiratory system 10. Optionally, the respiratory system can be configured to titrate the flow rate 6′ (see dotted curved line in FIG. 1) of gases accordingly to meet the instantaneous inspiratory demand of the patient while avoiding unnecessarily higher flow rates, insofar that they are not required or could be harmful to the patient.

Referring to FIG. 2B, the inspiratory demand 7 of a patient is met when the flow rate 6 provided by the respiratory system is equal to or greater (“meets”) than the instantaneous inspiratory demand flow rate 7 (“inspiratory demand”—see 6′ FIG. 1). For example, if the oxygen fraction at the patient's nose and/or mouth is equal to or about (“matches”) the gas-flow oxygen fraction, then it is determined that the gas-flow meets the patient's inspiratory demand. It follows that the peak inspiratory demand is met when the flow rate provided by the respiratory system is equal to or greater than the peak inspiratory demand flow rate 5 (“peak inspiratory demand”—see 6″ FIG. 1). The present embodiments (as per FIG. 3) can determine if the flow of gas meets inspiratory demand (either peak or instantaneous, depending on the configuration), and if not (as per FIG. 2A) will optionally alter operation to provide a flow of gas 6 to a patient at a flow rate 6′ high enough to meet the inspiratory demand 7 (preferably peak inspiratory demand 6″) of the patient, such that there is no or insignificant entrainment of ambient air as shown in FIG. 2B. In some embodiments a flow rate limit may be applied to the alteration of the flow rate by the respiratory system when there is entrainment of ambient of air by the patient. For example, the respiratory system may increase the flow rate of gases when entrainment of ambient air is determined but respiratory system may not continually increase the flow rate past a predetermined flow rate limit. This is to avoid excessively or unnecessarily high flow rates that might be detrimental to the patient.

Embodiments will now be described. Note, the embodiments will describe system (apparatus) and/or methods for meeting “inspiratory demand”, by way of example. This could refer to meeting “peak inspiratory demand” or meeting “instantaneous inspiratory demand” depending on the configuration. For meeting peak inspiratory demand, the oxygen concentration measurement at the nose and/or mouth is made at the time of peak inspiratory demand, or throughout the breath cycle (continuous or periodic determination). But these embodiments could equally be used to meet instantaneous inspiratory demand. To do that, the same apparatus/method is used, except that the oxygen concentration measurement at the nose and/or mouth is determined continuously or periodically throughout the breath cycle.

### One Embodiment

The respiratory system (can also be termed a “respiratory apparatus”) 10 for providing flow therapy or other therapy to a patient will be described in more detail in accordance with one embodiment. The respiratory system is configured for delivering a patient gas flow and determining whether the flow rate of the gas flow provided to the patient meets inspiratory demand of the patient. As a result, it can also modify the patient gas-flow flow rate to meet inspiratory demand. The respiratory system may be used for any suitable oxygenation purpose including, without limitation, pre-oxygenation during an anaesthetic procedure (e.g. anesthesia or sedation), after anaesthetic or sedative agents are administered to a patient during an anaesthetic procedure (e.g. anaesthesia or sedation; as per the disclosure of PCT applications WO2016/157102, and WO2016/133406 (US equivalents being US20180280641 and US20180126110 respectively) which are incorporated herein in their entirety) for example, high flow respiratory support, high flow therapy, ventilation, provision of high flow gas-flows or anywhere else where monitoring of whether the patient's inspiratory demand is being met (whether by the respiratory system gas flow or entrained air) is required.

The respiratory system comprises a flow source 50 for providing a high flow gas 31 such as oxygen, or a mix of oxygen and one or more other gases. Alternatively, the respiratory system can have a connection for coupling to a flow source. As such, the flow source might be considered to form part of the respiratory system or be separate to it, depending on context, or even part of the flow source forms part of the respiratory system, and part of the flow source falls outside of the respiratory system. In short, the system can have:
a flow source
humidifier for humidifying the gas-flow,
inspiratory tube,
conduit (e.g. dry line or heated breathing tube),
patient interface,
pressure relief valve
filter

The system will be described in more detail.

The flow source could be an in-wall supply of oxygen, a tank of oxygen 50A, a tank of other gas and/or a high flow apparatus with a flow generator 50B. FIG. 3 shows a flow source 50 with a flow generator 50B, with an optional air inlet 50C and optional connection to an O2 source (such as tank or O2 generator) 50A via a shut off valve and/or regulator and/or other gas flow control 50D, but this is just one option. The flow generator 50B can control flows delivered to the patient 16 using one or more valve, or optionally the flow generator 50B can comprise a blower. The flow source could be one or a combination of a flow generator 50B, O2 source 50A, air source 50C as described. The flow source 50 is shown as part of the respiratory system 10, although in the case of an external oxygen tank or in-wall source, it may be considered a separate component, in which case the respiratory system has a connection port to connect to such flow source. The flow source provides a (preferably high) flow of gas that can be delivered to a patient via a delivery conduit, and patient interface 51.

The patient interface 51 may be an unsealed (non-sealing) interface (for example when used in high flow therapy) such as a non-sealing nasal cannula, or a sealed (sealing) interface (for example when used in CPAP) such as a nasal mask, full face mask, or nasal pillows. In some embodiments, the patient interface 51 is a non-sealing patient interface which would for example help to prevent barotrauma (e.g. tissue damage to the lungs or other organs of the respiratory system due to difference in pressure relative to the atmosphere). In some embodiments, the patient interface 51 is a sealing mask that seals with the patient's nose and/or mouth. The patient interface may be a nasal cannula with a manifold and nasal prongs, and/or a face mask, and/or a nasal pillows mask, and/or a nasal mask, and/or a tracheostomy interface, or any other suitable type of patient interface. The flow source could provide a base gas flow rate of between, e.g. 0.5 litres/min and 375 litres/min, or any range within that range, or even ranges with higher or lower limits, as previously described. Details of the ranges and nature of flow rates will be described later.

A humidifier 52 can optionally be provided between the flow source 50 and the patient to provide humidification of the delivered gas. One or more sensors 53A, 53B, 53C, 53D such as flow, oxygen fraction, pressure, humidity, temperature or other sensors can be placed throughout the system and/or at, on or near the patient 16. Alternatively, or additionally, sensors from which such parameters can be derived could be used. In addition, or alternatively, the sensors 53A-53D can be one or more physiological sensors for sensing patient physiological parameters such as, heart rate, oxygen saturation, partial pressure of oxygen in the blood, respiratory rate, partial pressure of CO2 in the blood. Alternatively, or additionally, sensors from which such parameters can be derived could be used. Other patient sensors could comprise EEG sensors, torso bands to detect breathing, and any other suitable sensors. In some configurations the humidifier may be optional, or it may be preferred due to the advantages of humidified gases helping to maintain the condition of the airways. One or more of the sensors might form part of the respiratory system, or be external thereto, with the respiratory system having inputs for any external sensors. The sensors can be coupled to or send their output to a controller 19.

A sensor 14 is provided for measuring the oxygen fraction of air the patient inspires. This can be placed on the patient interface 51, for example, to measure or otherwise determine the fraction of oxygen proximate (at/near/close to) the patient's mouth and/or nose. The output from the sensor 14 is sent to a controller 19 to assist control of the respiratory system to determine if the peak inspiratory demand is being met, and alter operation accordingly. The controller 19 is coupled to the flow source 50, humidifier 52 and sensor 14. It controls these and other aspects of the respiratory system to be described below. The controller can operate the flow source to provide the delivered flow of gas at a desired flow rate high enough to meet peak inspiratory demand. In an alternative, the sensor 14 might convey measurements of oxygen fraction at the patient mouth and/or nose to a user, who then inputs the information to the respiratory apparatus/controller. Any disclosure/embodiment hereinafter could be read as having that alternative, where appropriate.

The controller 19 is also configured to operate the respiratory system so that the patient gas flow has a flow rate with an oxygen fraction that meets the patient's requirements and provides the required therapy. The oxygen fraction may be a known oxygen fraction. For example, where pre-oxygenation of a patient prior to administration of anaesthesia is desired, the controller 19 can operate the respiratory system to provide a gas flow with an oxygen fraction of at or about 100%. In another example, where sedation of a patient is desirable, the controller 19 can operate the respiratory system to provide a gas flow with an oxygen fraction of about 21% or more during the sedation procedure. Preferably, the oxygen fraction of the gas flow provided during a sedation procedure is more than 21%, for example about 30% or about 50% or more. If the patient becomes apnoeic during the sedation procedure, the controller 19 or clinician can adjust the oxygen fraction of the gas flow to anywhere between about 21% and about 100%. Preferably, the controller 19 will increase the oxygen fraction in the gas flow, preferably to an oxygen fraction greater than a previous oxygen fraction, but this could be done manually. It can do this in any suitable way, for example by controlling a valve coupled to the O2 source to increase/decrease the amount of O2 relative to ambient gas flow to control the proportion(concentration) of O2 in the total gas flow.

An input/output interface (user interface) 54 (such as a display and/or input device) is provided. The input device is for receiving information from a user (e.g. clinician or patient) that can be used for determining oxygenation requirements, anaesthetic gas agent and/or CO2 detection. For example, but not limiting, the user interface can be used input the oxygen concentration information from the patient mouth and/or nose to the respiratory apparatus/controller. The respiratory system can also be operated to determine dose/oxygenation requirements (hereinafter “oxygen requirements”) of a patient for/in relation to anaesthesia (that is, the oxygen requirements pre-anaesthesia during a pre-oxygenation phase and/or the oxygen requirements during anaesthesia—which might include when the patient is apnoeic or when the patient is breathing), as well as after such a procedure, which may include the extubation period. The respiratory system 10 is also configured to adjust and provide high flow gas to a patient for the purposes of anaesthesia and adjust the parameters of the high flow gas (such as pressure, flow rate, volume of gas, gas composition) delivered to the patient as required to meet oxygenation requirements, for example based on a determined oxygen fraction at the patient's nose and/or mouth. The respiratory system also comprises a display which can be part of the I/O for displaying the estimated measure of the gas parameter of the exhaled gas flow, as a graph, digital readout or any other suitable means. The controller can determine if inspiratory demand is being met or not being met, and output an indication of that on the user interface.

The sensor(s), controller 19 and/or any other components that measure the oxygen fraction can be considered a “detection system”. As described above, the detection system is integrated into the respiratory system 10, with aspects of the detection system being used for other functionality also. However, it could be appreciated that there could be a separate detection system, either integrated with the respiratory system or separate to it.

The respiratory system 10 could be an integrated or a separate component-based arrangement, generally shown in the dotted box in FIG. 3. In some configurations, the respiratory system could be a modular arrangement of components. Furthermore, the respiratory system may just comprise some of the components shown, not necessarily all are essential. Also, the conduit and patient interface do not have to be part of the system, and could be considered separate. Hereinafter it will be referred to as respiratory system, but this should not be considered limiting. Respiratory system will be broadly considered herein to comprise anything that provides a flow rate of gas to a patient with which a detection system can be used to determine if the flow rate of gas meets inspiratory demand.

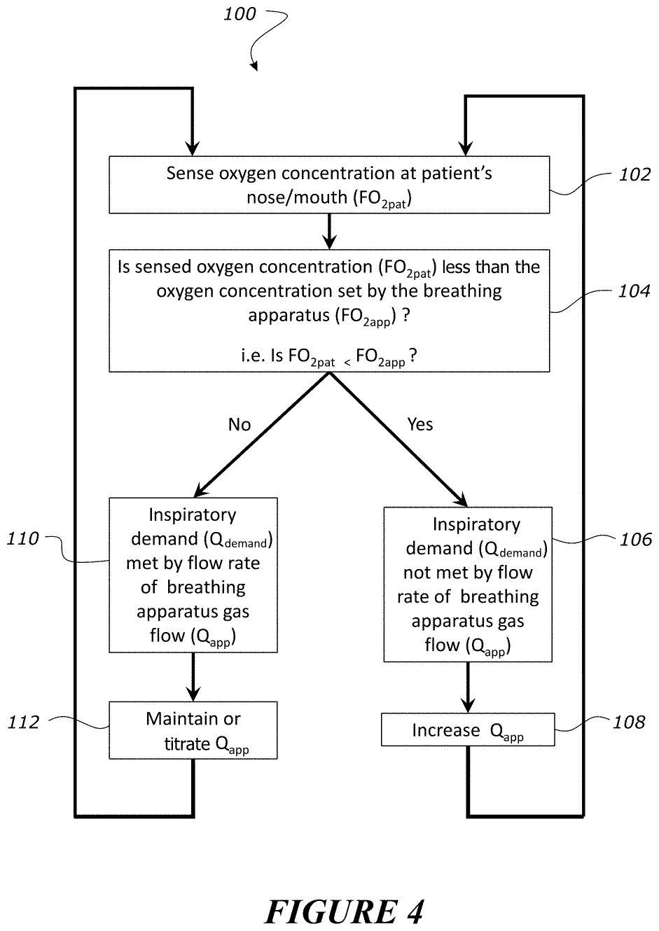

FIG. 4 shows a flow diagram showing the method steps 100 the controller in respiratory system 10 is configured to operate. The flow diagram shows the more general example of meeting instantaneous inspiratory demand. In one example, this could be peak inspiratory demand. At step 102, the controller receives a signal from the sensor 14 (directly or via a user) that is (or is indicative of) the oxygen concentration of the air the patient is inspiring FO₂ₚₐₜ. At step 104, the controller compares the sensed (received directly or via a user) oxygen concentration FO₂ₚₐₜ against the oxygen concentration of the respiratory system gas flow FO₂ₐₚₚ. If the sensed oxygen concentration FO₂ₚₐₜ is less than the oxygen concentration of the respiratory system FO₂ₐₚₚ, the controller progresses to step 106 and determines that the inspiratory demand Q_{demand} is not being met by the flow rate of the respiratory system gas flow Qₐₚₚ, and proceeds to increase the flow rate of the respiratory system gas flow Qₐₚₚ at step 108, before repeating step 102.

Adjustment of the flow rate of gas flow to the patient will now be described. If the controller determines at step 106 that the flow rate should be increased, the controller alerts the clinician of the need to increase the flow rate, and/or optionally a flow rate to increase to or a flow rate amount to increase by. This can be done by sounding an alarm indicative of a need to increase the flow rate and/or displaying information, such as a flow rate or flow rate increase amount on display. The clinician can then operate the apparatus to increase the flow rate accordingly. This can be done by sending a user input to the controller to operate the apparatus to increase the flow rate, at step 108. Alternatively, the controller determines the flow rate to increase to or flow rate amount to increase to, and operates the flow source 50 to reach or increase the flow rate accordingly.

Optionally, the flow rate to increase to can be determined as follows.

The measured FiO2 is given by equation (1):

$$Fi O 2 = \frac{0.21 * ambient flow rate + 1 x apparatus flow rate}{ambient flow rate + a p p a ratus flow rate}$$

Where ambient flow rate is the flow rate of entrained ambient air by the patient, and the apparatus flow rate is the flow rate of gas flow being delivered by the apparatus to the patient.

Rearranging from ambient flow rate we get equation (2):

$$ambient flow rate = \frac{apparatus flow rate ( 1 - Fi O 2 )}{Fi O 2 - 0.21}$$

Ambient flow rate can be calculated from (2) using knowledge of the apparatus flow rate.

Inspiratory flow rate is given by equation (3):

Instantaneous Insp Flow rate=apparatus flow rate+ambient flow rate

The instantaneous inspiratory flow rate can be calculated from equation (3) by using the ambient flow rate calculated in equation (2) and knowledge of the apparatus flow rate. The apparatus flow rate delivered to the patient can then be changed by increasing to the calculated instantaneous inspiratory flow.

Irrespective of how it is determined, as an example, the gas-flow flow rate is increased to a flow rate of more than 20 litres per minute, optionally between about 20 litres per minute and about 90 litres per minute, or between about 40 litres per minute and about 70 litres per minute. Also as an example, the gas-flow flow rate is increased by increments of more than 0 litres per minute, optionally about 1 or more litres per minute, optionally about 5 or more litres per minute, or optionally about 10 or more litres per minute. In some embodiments, the flow rate increments are stepped increments and/or continual increments.

On the other hand, if the sensed (or otherwise determined) oxygen concentration FO₂ₚₐₜ matches (e.g. equal, at, close to or near) the oxygen concentration of the respiratory system FO₂ₐₚₚ, the controller progresses to step 110 and determines that the inspiratory demand Q_{demand} is being met (or possibly exceeded) by the flow rate of the respiratory system gas flow Qₐₚₚ, and that the flow rate should be maintained; or if titration to the inspiratory flow is required, determines to decrease the flow rate of the respiratory system gas flow Qₐₚₚ at step 112, before repeating step 102.

If it is decided to titrate the flow rate provided to the patient to get closer to the actual inspiratory demand (rather than exceeding it), the following happens. If the controller determines at step 110 that the flow rate should be decreased, the controller alerts the clinician of the need to decrease the flow rate, and/or optionally a flow rate to decrease to or a flow rate amount to decrease by. This can be done by sounding an alarm indicative of a need to decrease the flow rate. and/or displaying information, such as a flow rate or flow rate decrease amount on display The clinician can then operate the respiratory system to decrease the flow rate accordingly. This can be done by sending a user input signal to the controller to operate the apparatus to decrease the flow rate, at step 112. Alternatively, the controller determines the flow rate to decrease to or flow rate amount to decrease by, and operates the flow source 50 to decrease the flow rate accordingly. In some embodiments, the gas-flow flow rate is decreased to a flow of more than 20 litres per minute, optionally between about 20 litres per minute and about 90 litres per minute, or between about 40 litres per minute and about 70 litres per minute. Also as an example, the gas-flow flow rate is decreased by decrements of more than 0 litres per minute, optionally about 1 or more litres per minute, optionally about 5 or more litres per minute, or optionally about 10 or more litres per minute.

In some embodiments, the flow rate decrements are stepped decrements and/or continual decrements. Once the flow rate is decreased (by either means), another measurement is taken. If the measured oxygen concentration is still equal to the delivered concentration, inspiratory demand is still at least being met (or possibly exceeded), the flow rate is decreased again as per above. This can be continued until the measured oxygen concentration is less than that delivered oxygen concentration, which indicates that patient is entraining air, and so this will be just below the point of meeting inspiratory demand. Optionally, at this point the flow rate can be maintained, or increased again to finally settle on or close to the inspiratory demand. Therefore, the flow rate is decreased until the oxygen concentration level reaches or is close to the optimal value, indicating that inspiratory flow is just being met. There could be a constant feedback loop to try to retain the flow rate at as close as possible to the inspiratory demand. In an alternative, the gas-flow flow rate is maintained until it is determined that the patient inspiratory demand is not being met.

When titrating as above, there is a point at which further decrease in flow rate is not implemented and the flow rate is maintained or increased. There are various ways. For example, the controller can be configured to continually prompt the clinician to operate the respiratory system to decrease the flow rate or the controller operates the flow source 50 to decrease the flow rate until the difference between the oxygen fraction of the delivered gas flow and the oxygen fraction at the mouth and/or nose exceeds a threshold (that is, the oxygen fraction at the nose and/or mouth is more than a predetermined threshold lower than the oxygen fraction of the delivered gas flow). At this point, the decrease in flow rate ceases.

So, as an example, the controller is configured to prompt the clinician or control the flow source to continue decreasing the flow rate while the difference between the delivered oxygen fraction and the oxygen fraction at the mouth and/or nose is less than e.g. 5% oxygen fraction. If the difference between the delivered oxygen fraction and oxygen fraction at the mouth and/or nose exceeds 5% oxygen fraction, the controller determines that the flow rate should not decrease any further, but instead determines that the flow rate should be maintained, (or increased again if implementing a feedback loop).

In one option, no action is taken, until a sufficient proportion of previously measured breath cycles have entrainment of air taking place. Only then does the controller prompt the clinician, or controls the flow source, to increase the flow rate. For example, the respiratory system maintains the flow rate for a set number of breath cycles (e.g. 5) and observes if there is any entrainment (that is oxygen fraction at the patient is less than gas-flow oxygen fraction) during any one of the cycles. If a sufficient set number of cycles show entrainment, the controller prompts the clinician or controls the flow source to increase the flow rate, and if an acceptable number of cycles (e.g. 2 or less) show entrainment, the apparatus could determine that peak inspiratory demand is generally met over that set number of cycles and maintain the flow rate of the delivered gases. The set number of cycles and threshold of acceptable cycles can be pre-determined or determined by the user.

In one example determination that patient inspiratory demand is not being met comprises the controller: monitoring a set number of previous patient breath cycles,
determining a number of previous patient breath cycles that have entrainment of ambient air, and comparing the number of previous patient breath cycles having entrainment of ambient air against a set threshold. The patient inspiratory demand is not met if the number of previous patient breath cycles having entrainment of ambient air exceeds a set threshold.

It should be noted here that the sensor 14 might be measuring the oxygen concentration proximate the patient's nose/mouth continuously/periodically throughout the entire breath cycle such that it measures the concentration at times other than at peak patient inspiration. If this is the case, even at apparatus flow rates less than peak inspiratory demand, the controller might determine that the oxygen concentration is sufficient and therefore the flow rate is sufficient for meeting instantaneous inspiratory demand. However, this will correct itself because as the patient inspiratory demand peaks, the sensor 14 will re-measure and once at peak will be able to determine if peak inspiratory demand is being met.

The pattern of Qₚₑₐₖ can be displayed on the user interface 54 and observed to provide diagnostic information, for example if the patient's breathing is changing and how it is changing, e.g. respiratory rate is reduced over time.

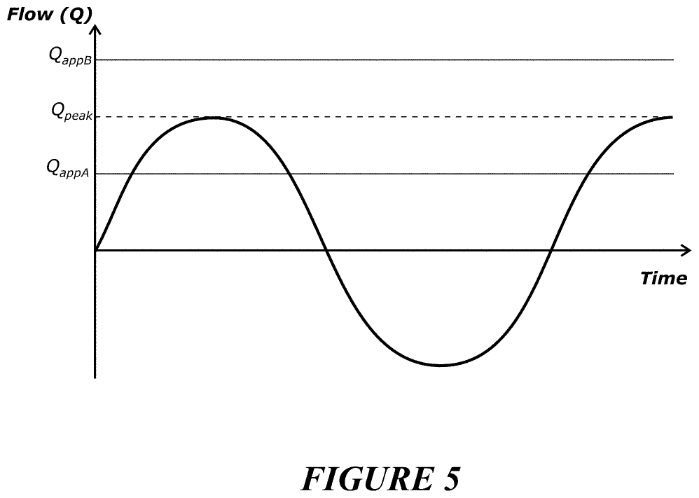

Operation of the respiratory system will now be described with reference to the apparatus of FIG. 3 and the flow diagram of FIG. 4. The apparatus is operated in the usual way for high flow therapy by controller receiving inputs from various sensors and other inputs, determining the flow rate and oxygen concentration required by the patient, and controlling the flow generator to provide a flow of gas with the desired flow rate and oxygen concentration. The oxygen concentration may be set by a user, or set by the controller depending on the therapy mode of the apparatus. Therefore, the high flow therapy device will provide a flow of gas at determined flow rate with a determined O2 concentration. At this point, the flow rate may or may not meet the (peak) inspiratory demand. FIG. 5 shows an example of where the flow rate Q_{appA} meets inspiratory demand over a portion of the patient's breath but does not meet peak inspiratory demand Qₚₑₐₖ, and where flow rate Q_{appB} meets inspiratory demand and peak inspiratory demand Qₚₑₐₖ.

The controller receives input from the sensor 14 (directly or via a user), which measures the concentration of oxygen at the patient's nose/mouth, step 102. The sensor might provide information of an oxygen fraction to the controller, or the controller might determine an oxygen fraction from the sensor. Alternatively, there might not be any actual determination of an oxygen fraction, but another related parameter from which the controller can determine a relative relationship between an oxygen fraction of the gas flow and an oxygen fraction at the patient's nose/mouth. Next, the controller determines whether the oxygen concentration at the patient's nose/mouth (as measured by the sensor) is greater than, equal to or less than the oxygen concentration of the flow of gas, step 104. If the oxygen concentration at the patient's nose/mouth is less than the oxygen concentration in the flow of gas (see Q_{appA} in FIG. 5), the controller will determine that peak inspiratory demand is not being met, step 106 by the gas-flow. This is because it is presumed, that because the oxygen concentration at the nose/mouth is less than that provided in the flow of gas, that the patient must be entraining ambient air. In this case, the controller can alter the operation of the respiratory system (or the clinician can) to remedy the situation, for example by controlling the valves and/or blower in the flow generator 50B to increase the flow rate of the flow gas provided by the respiratory system, step 108. Alternatively, the controller via the user interface could prompt a clinician to manually alter the flow rate.

If the oxygen concentration at the patient's nose/mouth is equal to or substantially equal to the oxygen concentration in the flow of gas (see Q_{appB} in FIG. 5), the controller will determine that peak inspiratory demand is being met, step 110 (or possibly being exceeded). This is because it is presumed, that because the oxygen concentration at the nose/mouth is equal to that provided in the flow of gas, that the patient is likely not to be entraining ambient air. In this case, the controller can either do nothing, or alter the operation of the flow therapy apparatus to lower the flow rate provided to the patient (in the instance where it may be considered that there is too much flow, and it is not necessary to maintain that flow rate), for example by controlling the flow generator to decrease the flow rate of the flow gas provided by the respiratory system, step 112.

It should be noted that the controller does not necessarily determine what the inspiratory demand is, but it is possible to do so and to convey that.

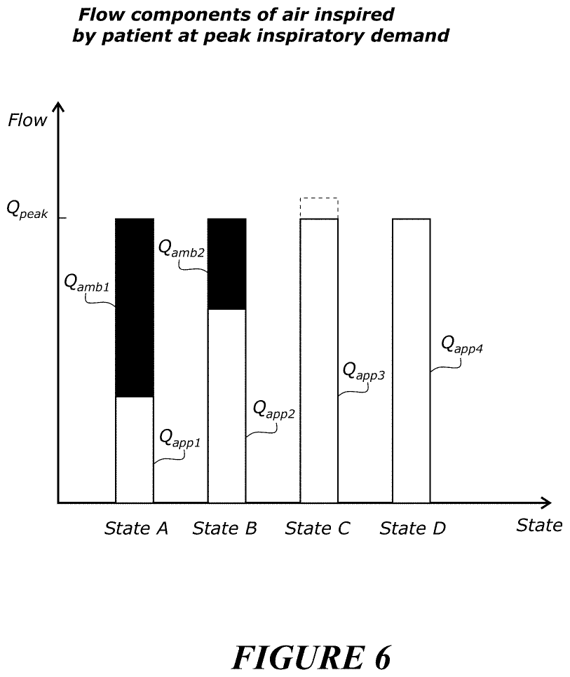

Referring to FIGS. 6 and 7, these Figures show how the controller of the respiratory system 10 determines whether the respiratory system is providing sufficient flow of gas to meet inspiratory demand, and optionally titrates the flow rate of the gas flow being delivered to the patient to provide a desired flow rate high enough to meet instantaneous or peak inspiratory demand. FIG. 6 is a bar graph showing how the flow composition of the patient inspiration (Qₚₐₜᵢₑₙₜ) varies based on flow rate of the gas flow provided by the respiratory system (Qₐₚₚ). FIG. 7 is a line graph showing how the oxygen concentration of air the patient inspires (FiO₂) varies depending on the flow rate of the gas flow the respiratory system provides (Qₐₚₚ). States A-D as shown in FIGS. 6 and 7 help show how operation of the respiratory system changes, while FIG. 4 shows the steps 100 the controller 14 takes to transition from state A through to state D.

At state A, respiratory system 10 initially provides a flow of gas with an oxygen concentration FO₂ₐₚₚ greater than the oxygen concentration of ambient air FO_{2amb}, and at a first respiratory system flow rate of Qₐₚₚ₁. The first respiratory system flow rate Qₐₚₚ₁ is insufficient for meeting peak inspiratory demand Qₚₑₐₖ so ambient air is entrained into the patient's airways at a first ambient flow rate Q_{amb1}, when the patient is at peak inspiration. The flow rate the patient inspires Qₚₐₜᵢₑₙₜ is at the peak inspiratory demand Qₚₑₐₖ, which in this situation is a combination of the first respiratory system flow rate Qₐₚₚ₁ and the first ambient flow rate Q_{amb1}. The entrainment of ambient air dilutes the oxygen concentration of the gases the patient inspires such that the patient inspires gases at a first oxygen concentration FO₂ₚₐₜ₁ that is greater than the oxygen concentration of ambient air FO_{2amb} but less than the desired oxygen concentration of the gas flow the respiratory system provides FO₂ₐₚₚ.

This means at step 102, the sensor 14 senses the first oxygen concentration at the patient's nose/mouth FO₂ₚₐₜ₁. At step 104, the controller 19 determines the first oxygen concentration FO₂ₚₐₜ₁ is less than the oxygen concentration of the respiratory system gas flow FO₂ₐₚₚ, and determines at step 106 that the flow rate of the respiratory system gas flow Qₐₚₚ₁ is insufficient for meeting peak inspiratory demand Qₚₑₐₖ. The controller 19 increases the flow rate of the respiratory system gas flow Qₐₚₚ at step 108 to a second respiratory system flow rate of Qₐₚₚ₂. This creates state B.

At state B, respiratory system 10 now provides a flow of gas at a second respiratory system flow rate of Qₐₚₚ₂. Despite being an increase from the first respiratory system flow rate Qₐₚₚ₁, the second respiratory system flow rate Qₐₚₚ₂ is still insufficient for meeting peak inspiratory demand Qₚₑₐₖ so ambient air is entrained into the patient's airways at a second ambient flow rate Q_{amb2}, when the patient is at peak inspiration. The flow rate the patient inspires Qₚₐₜᵢₑₙₜ is at the peak inspiratory demand Qₚₑₐₖ, which in this situation is a combination of the second respiratory system flow rate Qₐₚₚ₂ and the second ambient flow rate Q_{amb2}. The entrainment of ambient air dilutes the oxygen concentration of the gases the patient inspires such that the patient inspires gases at a second oxygen concentration FO₂ₚₐₜ₂ that is greater than the oxygen concentration of ambient air FO_{2amb}, greater than the first oxygen concentration FO₂ₚₐₜ₁, but less than the oxygen concentration of the gas flow the respiratory system provides FO₂ₐₚₚ. This means at step 102, the sensor 14 senses the second oxygen concentration at the patient's nose/mouth FO₂ₚₐₜ₂. At step 104, the controller 19 determines the second oxygen concentration FO₂ₚₐₜ₂ is less than the oxygen concentration of the respiratory system gas flow FO₂ₐₚₚ, and determines at step 106 that the flow rate of the respiratory system gas flow Qₐₚₚ₁ is insufficient for meeting peak inspiratory demand Qₚₑₐₖ. The controller 19 increases the flow rate of the respiratory system gas flow Qₐₚₚ at step 108 to a third respiratory system flow rate of Qₐₚₚ₃. This leads to state C.

At state C, the respiratory system 10 now provides a flow of gas at a third respiratory system flow rate of Qₐₚₚ₃, which is an increase from the first respiratory system flow rate Qₐₚₚ₁ and an increase from the second respiratory system flow rate Qₐₚₚ₂. The third respiratory system flow rate Qₐₚₚ₃ is at least equal to or greater than (see dotted portion) the peak inspiratory demand Qₚₑₐₖ, which means the third respiratory system flow rate Qₐₚₚ₃ meets peak inspiratory demand Qₚₑₐₖ (meaning Qₐₚₚ₃ is a flow rate at least as great as peak inspiratory demand, if not greater) such that the flow rate the patient inspires Qₚₐₜᵢₑₙₜ is equal to or less than the third respiratory system flow rate Qₐₚₚ₃, and there is no entrainment of ambient air i.e. Q_{amb}=0. The patient inspires gases at a third oxygen concentration FO₂ₚₐₜ₃ that is equal to the oxygen concentration of the gas flow the respiratory system provides FO₂ₐₚₚ. This means at step 102, the sensor 14 senses the third oxygen concentration at the patient's nose/mouth FO₂ₚₐₜ₃.

At step 104, the controller 19 determines the third oxygen concentration FO₂ₚₐₜ₃ is equal or substantially equal to the oxygen concentration of the respiratory system gas flow FO₂ₐₚₚ, and determines at step 110 that the flow rate of the respiratory system gas flow Qₐₚₚ₃ is sufficient for at least meeting peak inspiratory demand Qₚₑₐₖ. At this point, the controller 19 maintains a respiratory system flow rate of Qₐₚₚ₃.

In state C, it's possible that the flow rate of the respiratory system gas flow Qₐₚₚ₃ may actually be exceeding peak inspiratory demand Qₚₑₐₖ. In this case, it may not be desirable to maintain the flow rate, as it is unnecessarily high. Therefore, as an alternative (because it's possible the flow rate of the respiratory system gas flow Qₐₚₚ₃ may in fact be exceeding peak inspiratory demand Qₚₑₐₖ at state C.), and as previously described, the controller 19 uses a feedback control to decrease the flow rate of the respiratory system gas flow Qₐₚₚ at step 112 to a fourth respiratory system flow rate of Qₐₚₚ₄ in order to drop the Qₐₚₚ so it substantially meets peak inspiratory flow. This leads to (test) state D. In state D, the respiratory system 10 provides a flow of gas at a fourth respiratory system flow rate of Qₐₚₚ₄, which is greater than the first and second respiratory system flow rates Qₐₚₚ₁, Qₐₚₚ₂, but less than the third respiratory system flow rate Qₐₚₚ₃. The fourth respiratory system flow rate Qₐₚₚ₄ is substantially equal to the peak inspiratory demand Qₚₑₐₖ, although this could still be greater or less than the peak inspiratory demand Qₚₑₐₖ. This means the fourth respiratory system flow rate Qₐₚₚ₄ substantially meets peak inspiratory demand Qₚₑₐₖ such that the flow rate the patient inspires Qₚₐₜᵢₑₙₜ is substantially equal to the fourth respiratory system flow rate Qₐₚₚ₄, (for example, they are equal or equal to within a tolerance, such as 5%, 4%, 3%, 2%, 1% or some other tolerance) and there is no substantial entrainment of ambient air i.e. Q_{amb}=0. In some embodiments, the tolerance may be predetermined or may be determined by a user. The patient inspires air at a fourth oxygen concentration FO₂ₚₐₜ₄ that is substantially equal to the oxygen concentration of the gas flow the respiratory system provides FO₂ₐₚₚ. The fourth respiratory system flow rate Qₐₚₚ₄ is substantially the minimum flow rate the respiratory system gas flow can provide while ensuring the patient is properly oxygenated (by ensuring the patient actually inspires an oxygen fraction set by the respiratory system).

To achieve state D from state C (where the flow rate might exceed inspiratory demand), the flow rate can be titrated so that the fourth respiratory system flow rate Qₐₚₚ₄ is equals to the peak inspiratory demand Qₚₑₐₖ. In general terms, a closed loop or other control can be implemented, whereby the flow rate is decreased and the oxygen fraction measured to see if the flow rate still meets inspiratory demand until the lowest flow rate that meets inspiratory demand is found.

For example, step up or step down in flow rates can be set, and can be reduced as the flow rate Qₐₚₚ converges towards the peak inspiratory demand Qₚₑₐₖ. Optionally, there could be additional states that follow state D.

Alternatively, if there is a decrease in flow rate from third respiratory system flow rate of Qₐₚₚ₃ of state C, such that the decrease in flow rate “undershoots” the flow rate of state D, then the patient's peak inspiratory demand Qₚₑₐₖ would no longer be met and entrainment of ambient air would then be occurring. In this situation, the controller is able to determine that the patient is inspiring at an oxygen concentration FO₂ₚₐₜ less than the oxygen concentration of the gas flow provided by the respiratory system FO₂ₐₚₚ, and therefore determine that peak inspiratory demand Qₚₑₐₖ is no longer being met. That is, at step 102, the sensor 14 senses the oxygen concentration at the patient's nose/mouth FO₂ₚₐₜ. At step 104, the controller 19 determines the second oxygen concentration FO₂ₚₐₜ is less than the oxygen concentration of the respiratory system gas flow FO₂ₐₚₚ, and determines at step 106 that the flow rate of the respiratory system gas flow Qₐₚₚ is insufficient for meeting peak inspiratory demand Qₚₑₐₖ. The controller 19 increases the flow rate of the respiratory system gas flow Qₐₚₚ at step 108.

Other titration control systems to achieve state D are possible and the above are examples only. Also, it is not essential to titrate to state D. It would be possible to stay in state C where the flow rate might be above the inspiratory demand.

In the embodiments above, the apparatus can also be configured to determine whether a patient has stopped or reduced breathing. This is based on the premise that if a zero (or low) flow rate of gas is provided to a patient, yet the apparatus still determines that inspiratory demand is being met, this could be an indication that the patient may not be breathing or may be substantially diminished breathing. If they were breathing or have undiminished breathing, at zero flow rate they would likely be entraining ambient air, and the apparatus would determine that inspiratory demand is not being met.

In this embodiment, the system could supply a flow of gases at a specific flow rate with a known oxygen fraction (e.g. 100%). It could do the check for whether inspiratory demand is being met in the usual way. If inspiratory demand is being met, this could be due to the fact the flow rate is actually meeting inspiratory demand. However, it could also be because the patient is not breathing or have diminished breathing. As a test, the apparatus can be configured to reduce the flow rate towards zero, or towards some low threshold, and check if inspiratory demand is being met. If it is still being met, this would be implausible if the patient was breathing or have undiminished breathing, because such a low flow rate could not possibly meet inspiratory demand. Therefore, it can be inferred that the patient is not breathing. An alarm and/or other suitable action can be taken.

Variations

Embodiments above refer to:
determining oxygen fraction at a patient's mouth and/or nose,
determining whether an oxygen fraction at a patient's mouth and/or nose equals, is less than or more than an oxygen fraction in a gas flow being delivered to the patient, and/or
determining whether an inspiratory demand is being met or not being met based on the oxygen fraction determined at a patient's mouth and/or nose.
Adjusting the flow rate to meet inspiratory demand

In any of the above, it may not be strictly necessary to actually determine one or any of those outcomes above or action the adjustment as described. For explanatory and conceptual purposes, in the present embodiments, aspects can be characterised as “determining”, but in reality no determination might actually take place or need to take place. Rather, more generally an indication of oxygen fraction at the patients' mouth and/or is used (be it in the form of signal, voltage, current, data/information, value, user input or the like) and comparison can be made to oxygen fraction in the gas flow, and an appropriate outcome or action (such as advising a user, patient or clinician of whether respiratory demand is being met or not and/or adjusting a flow rate) can be taken. As such, in more general terms, the present invention relates to using an oxygen fraction at a patient's mouth and/or nose (irrespective of how it is determined and provided—e.g. measured, sensed or otherwise ascertained) and then using that knowledge along with knowledge about the oxygen fraction being delivered to take an appropriate action as above based on any discrepancy (or otherwise) between the oxygen fraction of the patient's mouth and/or nose and the oxygen fraction in the gas flow giving an indication about entrainment of air, which can then lead to an indication about whether inspiratory demand is being met or not, which itself can lead to an indication about an action that might need to be taken to either alleviate the situation or otherwise take action that is suitable. It is not essential to adjust flow rate to completely meet inspiratory demand, but just know that there is a discrepancy and take some action to at least partially ameliorate the discrepancy. The present invention relates to this and the embodiments above are just some of the implementations of the invention, but should not be considered restrictive and are there to support the more general concept.

For example, reference to determining an oxygen fraction at a patient's mouth and/or nose can mean obtaining any sort of indication, be it directly received from output from a sensor in the form of a signal, data, current, voltage, value, information, or the like, or otherwise determined from such output from a sensor, (such as, but not only limited to, by input from a user).

But as noted, above, such determination might not occur at all, or the indication might not be a direct indication of oxygen fraction, but some proxy for it. For example, the indication might just be something such as signal, data, current, voltage, value, information, parameter from which oxygen fraction could be determined, but does not necessarily have to be. The indication might be sufficient in itself to make the correlation to what needs to happen next to meet inspiratory demand.

As an example, the gas flow is adjusted based on fraction at the patient's mouth and/or nose and the oxygen fraction of the gas flow. This could be in the form of a comparison between some parameter indicative of oxygen fraction at the patient's mouth and/or nose and some parameter indicative of the oxygen fraction of the gas flow. That could include a comparison of actual oxygen fraction at the patient's mouth and/or nose (however determined) and the actual oxygen fraction of the gas flow, but that is not the only indicative parameter and less direct parameters could be used. The comparison might look at respectively whether one of the parameters is higher, lower and/or the same as the other parameter. In some embodiments, comparisons might not even be required. Just knowledge of the oxygen fraction at the patient's mouth or nose (or some parameter indicative of it) and/or knowledge of the oxygen fraction of the gas flow (or some parameter indicative of it) without actual comparison of the two might be sufficient to implement embodiments of the invention, whether it is determining inspiratory demand, adjusting the flow rate, adjusting the flow rate to meet inspiratory demand or some combination thereof. It is possible that an adjustment might not even fully resolve any discrepancy in meeting inspiratory demand—rather, it just gets the flow rate closer to the patient's inspiratory demand. (Broadly, “meeting” can mean “getting closer in flow rate to the inspiratory demand” where that provides a benefit). The knowledge of the oxygen fraction at the patient's nose and/or mouth and an oxygen fraction of the gas flow might be used to adjust the gas flow in a manner that improves the situation without necessarily actually meeting inspiratory demand. But, the oxygen fraction at the patient's mouth and nose and its relationship to the oxygen fraction of the gas flow can be the basis for obtaining information for making decisions, and/or adjusting the operation of the system.

Furthermore, the pattern of Qₚₑₐₖ can be displayed and observed to provide patient, diagnostic, therapy or other relevant information, for example if the patient's breathing is changing and how it is changing, e.g. respiratory rate is reduced over time. This could be displayed on the user interface in the form of values and/or graphs or the like. For example, flow rate, inspiratory demand, entrainment flow rate or the like could be indicated in a suitable fashion.

1. A respiratory system, comprising:
a flow generator to provide a gas-flow to a patient, the gas flow comprising an oxygen fraction, and
a controller configured to:
receive input from a sensor,
adjust the gas-flow flow rate based on an oxygen fraction at the patient's nose and/or mouth.

2. A respiratory system, comprising:
a flow generator to provide a gas-flow to a patient, the gas flow comprising an oxygen fraction, and
a controller configured to:
receive input relating to oxygen fraction at an patient's nose and/or mouth,
adjust the gas-flow flow rate based on the oxygen fraction at the patient's nose and/or mouth.

3. A respiratory system according to claim 1 or 2 wherein the flow generator provides a high flow gas-flow.

4. A respiratory system according to claim 1, 2 or 3 wherein the controller is further configured to obtain an indication of the oxygen fraction at the patient's nose or mouth.

5. A respiratory system according to any preceding wherein the gas-flow rate is adjusted based on a relationship between the oxygen fraction at the patient's nose and/or mouth and the gas-flow oxygen fraction.

6. A respiratory system according to any claim 1, or 3 to 5 wherein the sensor is a O2 fraction sensor coupled to the controller.

7. A respiratory system according to any preceding claim further comprising one or more of:
a humidifier for humidifying the gas-flow,
inspiratory tube,
conduit (e.g. dry line or heated breathing tube),
patient interface,
pressure relief valve
filter.

8. A respiratory system according to any preceding claim wherein the controller is further configured to:
determine if the gas-flow meets inspiratory demand or does not meet inspiratory demand based on the oxygen fraction at the patient's mouth and/or nose.

9. A respiratory system according to any preceding claim wherein the controller is further configured to:
determine if the gas-flow meets inspiratory demand or does not meet inspiratory demand based on a relationship between oxygen fraction at the patient's mouth and/or nose and the gas-flow oxygen fraction.

10. A respiratory system according to claim 7 or 8 further comprising a user interface and the controller configured to convey to a user whether a patient is meeting or not meeting inspiratory demand.

11. A respiratory system according to any preceding claim wherein the oxygen fraction is:
at least more than about 21%, and optionally:
100%, or
between about 30% and about 50%.

12. A respiratory system according to any preceding claims wherein the flow rate is optionally the flow rate is about 20 litres per minute or more, or optionally between about 20 litres per minute and 90 litres per minute, or optionally between about 40 litres per minute and 70 litres per minute.

13. A respiratory system according to any preceding claims comprising or for use with a non-sealing patient interface, preferably a non-sealing nasal cannula.

14. A respiratory system according to claim 8 or 9 wherein whether the gas-flow meets the patient's inspiratory demand comprises comparing the oxygen fraction at the mouth and/or nose against the gas-flow oxygen fraction.

15. A respiratory system according to claim 8, 9 or 14 wherein, if the determined oxygen fraction at the patient's nose and/or mouth is less than the gas-flow oxygen fraction, then it is determined that the gas-flow does not meet the patient's inspiratory demand.

16. A respiratory system according to claim 8, 9 or 14 if oxygen fraction at the patient's nose and/or mouth is equal to or about (“matches”) the gas-flow oxygen fraction, then it is determined that the gas-flow meets or is close to the patient's inspiratory demand.

17. A respiratory system according to any one of claims 8 to 16 wherein if the inspiratory demand is not being met, the gas-flow flow rate is increased by the controller.

18. A respiratory system according to claim 17 wherein the gas-flow flow rate is increased to a flow rate of optionally about 20 litres per minute or more, or optionally between about 20 litres per minute and about 90 litres per minute, or optionally between about 40 litres per minute and about 70 litres per minute.

19. A respiratory system according to any one of claims 8 to 16 wherein if the inspiratory demand is being exceeded, the gas-flow flow rate is maintained or decreased by the controller.

20. A respiratory system according to any one of claims 8 to 19 wherein the gas-flow flow rate is maintained until it is determined that the patient inspiratory demand is not being met.

21. A respiratory system according to claim 8, 9 or 14, 15 wherein determination that patient inspiratory demand is not being met comprises the controller:
monitoring a set number of previous patient breath cycles,
determining a number of previous patient breath cycles that have entrainment of ambient air, and
comparing the number of previous patient breath cycles having entrainment of ambient air against a set threshold.

22. A respiratory system according to claim 21 wherein patient inspiratory demand is not met if the number of previous patient breath cycles having entrainment of ambient air exceeds a set threshold.

23. A respiratory system according to any preceding claim wherein system is operated to provide the gas-flow to a patient prior to an anaesthetic procedure, and the oxygen fraction of the gas-flow is 100%.

24. A respiratory system according to any one of claims preceding claim wherein the system is operated to provide the gas-flow to a patient during a sedation procedure, and the oxygen fraction of the gas-flow is about 21% or more.

25. A respiratory system to claim 24 wherein if the patient become apnoeic during the sedation procedure, the oxygen fraction of the gas-flow is between about 21% and about 100%.

26. A method of providing a flow of gas from a respiratory system to a patient, comprising the steps of:
providing a gas-flow to a patient comprising an oxygen fraction,
adjusting the gas-flow flow rate based on an oxygen fraction at the patient's nose and/or mouth.

27. A method of providing a flow of gas from a respiratory system to a patient, comprising the steps of:
providing a gas-flow to a patient comprising an oxygen fraction, and
determining from an oxygen fraction at the patient's mouth and/or nose whether the gas-flow meets the patient's inspiratory demand.

28. A method according to claim 26 or 27 wherein the gas-flow is high flow.

29. A method according to any one of claims 26 to 28 further comprising obtaining an indication of the oxygen fraction at the patient's nose or mouth.

30. A method according to any one of claims 26 to 29 wherein the gas-flow rate is adjusted based on a relationship between the oxygen fraction at the patient's nose and/or mouth and the gas-flow oxygen fraction.

31. A method according to any one of claims 27 to 30 wherein determining whether the gas-flow meets the patient's inspiratory demand is based on a relationship between the oxygen fraction at the patient's mouth and/or nose against the gas-flow oxygen fraction.

32. A method according to any one of claims 27 to 31 wherein, if the oxygen fraction at the patient's nose and/or mouth is less than the gas-flow oxygen fraction, then it is determined that the gas-flow does not meet the patient's inspiratory demand.

33. A method according to any one of claims 27 to 32 wherein, if the oxygen fraction at the patient's nose and/or mouth is equal to or about (“matches”) the gas-flow oxygen fraction, then it is determined that the gas-flow meets the patient's inspiratory demand.

34. A method according to any one of claims 26 to 33 wherein if the inspiratory demand is not being met, the gas-flow flow rate is increased.

35. A method according to claim 34 wherein the gas-flow flow rate is increased to a flow rate optionally of about 20 litres per minute or more, or optionally between about 20 litres per minute and about 90 litres per minute, or optionally between about 40 litres per minute and about 70 litres per minute.

36. A method according to any one of claims 26 to 35 wherein if the inspiratory demand is being exceeded, the gas-flow flow rate is maintained or decreased.

37. A method according to claim 36 wherein the gas-flow flow rate is maintained until it is determined that the patient inspiratory demand is not being met.

38. A method according to claim 37 wherein determination that patient inspiratory demand is not being met comprises the steps of:
monitoring a set number of previous patient breath cycles,
determining a number of previous patient breath cycles that have entrainment of ambient air, and
comparing the number of previous patient breath cycles having entrainment of ambient air against a set threshold.

39. A method according to claim 38 wherein patient inspiratory demand is not met if the number of previous patient breath cycles having entrainment of ambient air exceeds a set threshold.

40. A method according to any one of claims 26 to 39 wherein gas-flow is provided to the patient prior to an anaesthetic procedure, and the oxygen fraction of the gas-flow is 100%.

41. A method according to any one of claims 26 to 40 wherein gas-flow is provided to the patient during a sedation procedure, and the oxygen fraction of the gas-flow is about 21% or more.

42. A method according to claim 41 wherein if the patient becomes apenic during the sedation procedure, the oxygen fraction of the gas-flow is between about 21% and about 100%.

43. A respiratory system configured with a controller and flow generator to adjust the gas-flow flow rate based on an oxygen fraction at the patient's nose and/or mouth and oxygen fraction of the gas-flow to meet or get close to inspiratory demand.
